(19) United States
(12) Patent Application Publication
(10) Pub. No.: US 2026/0033769 A1
(43) Pub. Date: Feb. 5, 2026

(54) APPARATUS AND METHOD FOR AUTOMATIC BEAT DETECTION DURING ELECTROANATOMIC MAPPING

(71) Applicant: Anumana, Inc., Cambridge, MA (US)

(72) Inventors: Animesh Agarwal, San Mateo, CA (US); Rohit Jain, Danville, CA (US); Robert L. Lux, Park City, UT (US)

(73) Assignee: Anumana, Inc., Cambridge, MA (US)

(21) Appl. No.: 18/788,888

(22) Filed: Jul. 30, 2024

(51) Int. Cl. A61B 5/367; A61B 5/00; A61B 5/287; A61B 5/339; A61B 5/35; A61B 5/353; A61B 5/366

(52) U.S. Cl. CPC A61B 5/367; A61B 5/287; A61B 5/339; A61B 5/35; A61B 5/353; A61B 5/366; A61B 5/7207; A61B 5/7239; A61B 5/7246

(57) ABSTRACT

An apparatus and method for automatic beat detection during electro anatomical mapping. The apparatus includes at least a catheter comprising at least a transducer, at least a localization system, at least a processor and a memory communicatively connected to the at least a processor. The memory instructs the processor to receive the at least a potential signal from the at least a transducer and the at least a position signal from the at least a localization system, determine, using a beat detection algorithm, at least a beat template as a function of the at least a potential signal, compare the at least a beat template with the at least a potential signal over time, and detect beat timing as a function of the comparison of the at least a beat template and the at least a potential signal.

### FIELD OF THE INVENTION

The present invention generally relates to the field of cardiac electrophysiology. In particular, the present invention is directed to an apparatus and a method for automatic beat detection during electroanatomic mapping.

### BACKGROUND

Current systems for mapping cardiac rhythms face challenges in accurately detecting and mapping cardiac rhythms in real-time. During medical procedures, patients often experience multiple rhythm changes, complicating the mapping process. Operators decide on a rhythm to focus on at the start of the procedure, ensuring that the geometric map aligns with the chosen rhythm. Existing solutions struggle to extract relevant segments of real-time signals that match the desired rhythm, leading to inaccuracies in the mapping process.

Detection algorithms play a role in this context, yet many systems lack the capability to automatically and accurately detect beats and match them to predefined templates. This limitation affects the ability to build accurate geometric and electrical maps of the heart. Additionally, current methods often fail to handle continuous and discontinuous arrhythmias effectively, resulting in incomplete or imprecise mapping. There is a need for a robust, automatic algorithm that can detect beats in real-time, match them to user-defined templates, and integrate this information into a comprehensive map.

### SUMMARY OF THE DISCLOSURE

In an aspect, an apparatus for automatic beat detection during electroanatomic mapping includes at least a processor and a memory communicatively connected to the at least a processor. The memory contains instructions configuring the processor to receive the at least a potential signal from the at least a transducer and the at least a position signal from the at least a localization system, determine, using a beat detection algorithm, at least a beat template as a function of the at least a potential signal, compare, using the beat detection algorithm, the at least a beat template with the at least a potential signal over time, and detect, using the beat detection algorithm, beat timing as a function of the comparison of the at least a beat template and the at least a potential signal.

In another aspect, a method for automatic beat detection during electroanatomic mapping includes receiving the at least a potential signal from the at least a transducer and the at least a position signal from the at least a localization system, determining, using a beat detection algorithm, at least a beat template as a function of the at least a potential signal, comparing, using the beat detection algorithm, the at least a beat template with the at least a potential signal over time, and detecting, using the beat detection algorithm, beat timing as a function of the comparison of the at least a beat template and the at least a potential signal.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram of an apparatus for automatic beat detection during electroanatomic mapping;

FIG. 2A is an illustration of a 12-lead potential signal graph of unacceptable noise;

FIG. 2B is an illustration of a 12-lead potential signal graph of noise during an experiment;

FIG. 3 is an illustration of a 12-lead potential signal graph wherein a window is selected for a P template and a QRS template;

FIG. 4 is an illustration of an exemplary graph of P wave detection;

FIG. 5 is an illustration of an exemplary graph of QRS wave detection;

FIG. 6A is an illustration of detected beats with the time variable aligned;

FIG. 6B is an illustration of a single detected beat;

FIG. 7A-B is an illustration of a root mean square of 12-lead potential signal graph;

FIG. 8A-B is an illustration of a typical atrial flutter (AFL) in a 12-lead potential signal graph where a derivative of the potential signal for each lead is calculated and then summed;

FIG. 9A-B an illustration of a reverse typical atrial flutter (AFL) in a 12-lead potential signal graph where a derivative of the potential signal for each lead is calculated and then summed;

FIG. 10 is a block diagram of an exemplary method for automatic beat detection during electroanatomic mapping; and

FIG. 11 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to apparatus and methods for automatic beat detection during electroanatomic mapping. The apparatus includes at least a computing device comprised of a processor and a memory communicatively connected to the processor. The memory instructs the processor to receive the at least a potential signal from the at least a transducer and the at least a position signal from the at least a localization system. The processor determines, using a beat detection algorithm, at least a beat template as a function of the at least a potential signal. The processor compares, using the beat detection algorithm, the at least a beat template with the at least a potential signal over time. Additionally, the processor detects, using the beat detection algorithm, beat timing as a function of the comparison of the at least a beat template and the at least a potential signal.

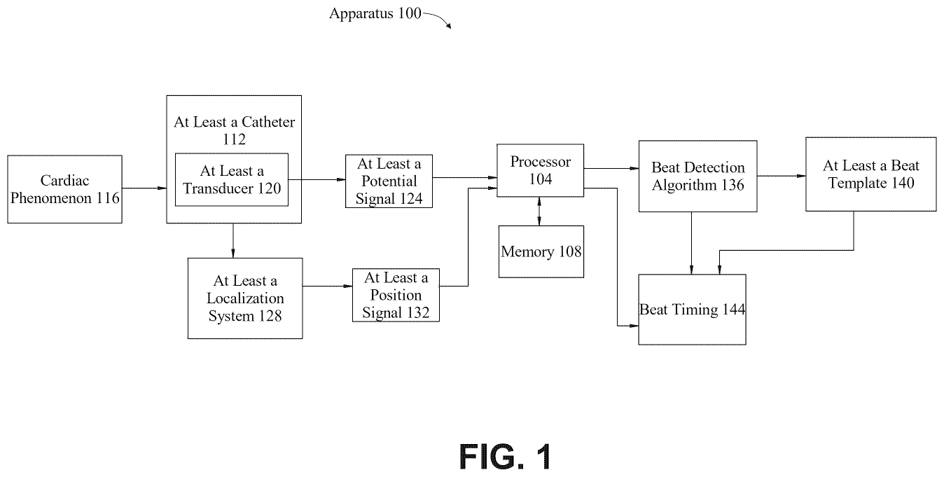

Referring now to FIG. 1, an exemplary embodiment of apparatus 100 for automatic beat detection during electroanatomic mapping is illustrated. Apparatus 100 may include a processor 104 communicatively connected to a memory 108. As used in this disclosure, “communicatively connected” means connected by way of a connection, attachment, or linkage between two or more relata which allows for reception and/or transmittance of information therebetween. For example, and without limitation, this connection may be wired or wireless, direct or indirect, and between two or more components, circuits, devices, systems, and the like, which allows for reception and/or transmittance of data and/or signal(s) therebetween. Data and/or signals there between may include, without limitation, electrical, electromagnetic, magnetic, video, audio, radio and microwave data and/or signals, combinations thereof, and the like, among others. A communicative connection may be achieved, for example and without limitation, through wired or wireless electronic, digital or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

With continued reference to FIG. 1, memory 108 may include a primary memory and a secondary memory. “Primary memory” also known as “random access memory” (RAM) for the purposes of this disclosure is a short-term storage device in which information is processed. In one or more embodiments, during use of the computing device, instructions and/or information may be transmitted to primary memory wherein information may be processed. In one or more embodiments, information may only be populated within primary memory while a particular software is running. In one or more embodiments, information within primary memory is wiped and/or removed after the computing device has been turned off and/or use of a software has been terminated. In one or more embodiments, primary memory may be referred to as “Volatile memory” wherein the volatile memory only holds information while data is being used and/or processed. In one or more embodiments, volatile memory may lose information after a loss of power. “Secondary memory” also known as “storage,” “hard disk drive” and the like for the purposes of this disclosure is a long-term storage device in which an operating system and other information is stored. In one or remote embodiments, information may be retrieved from secondary memory and transmitted to primary memory during use. In one or more embodiments, secondary memory may be referred to as non-volatile memory wherein information is preserved even during a loss of power. In one or more embodiments, data within secondary memory cannot be accessed by processor. In one or more embodiments, data is transferred from secondary to primary memory wherein processor 104 may access the information from primary memory.

Still referring to FIG. 1, apparatus 100 may include a database. The database may include a remote database. The database may be implemented, without limitation, as a relational database, a key-value retrieval database such as a NOSQL database, or any other format or structure for use as database that a person skilled in the art would recognize as suitable upon review of the entirety of this disclosure. The database may alternatively or additionally be implemented using a distributed data storage protocol and/or data structure, such as a distributed hash table or the like. The database may include a plurality of data entries and/or records as described above. Data entries in database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in database may store, retrieve, organize, and/or reflect data and/or records.

With continued reference to FIG. 1, apparatus 100 may include and/or be communicatively connected to a server, such as but not limited to, a remote server, a cloud server, a network server and the like. In one or more embodiments, the computing device may be configured to transmit one or more processes to be executed by server. In one or more embodiments, server may contain additional and/or increased processor power wherein one or more processes as described below may be performed by server. For example, and without limitation, one or more processes associated with machine learning may be performed by network server, wherein data is transmitted to server, processed and transmitted back to computing device. In one or more embodiments, server may be configured to perform one or more processes as described below to allow for increased computational power and/or decreased power usage by the apparatus computing device. In one or more embodiments, computing device may transmit processes to server wherein computing device may conserve power or energy.

Further referring to FIG. 1, apparatus 100 may include any “computing device” as described in this disclosure, including without limitation a microcontroller, microprocessor, digital signal processor (DSP) and/or system on a chip (SoC) as described in this disclosure. Apparatus 100 may include, be included in, and/or communicate with a mobile device such as a mobile telephone or smartphone. Apparatus 100 may include a single computing device operating independently, or may include two or more computing devices operating in concert, in parallel, sequentially or the like; two or more computing devices may be included together in a single computing device or in two or more computing devices. Apparatus 100 may interface or communicate with one or more additional devices as described below in further detail via a network interface device. Network interface device may be utilized for connecting processor 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Processor 104 may include but is not limited to, for example, a computing device or cluster of computing devices in a first location and a second computing device or cluster of computing devices in a second location. Apparatus 100 may include one or more computing devices dedicated to data storage, security, distribution of traffic for load balancing, and the like. Apparatus 100 may distribute one or more computing tasks as described below across a plurality of computing devices of computing device, which may operate in parallel, in series, redundantly, or in any other manner used for distribution of tasks or memory between computing devices. Apparatus 100 may be implemented, as a non-limiting example, using a “shared nothing” architecture.

With continued reference to FIG. 1, processor 104 may be designed and/or configured to perform any method, method step, or sequence of method steps in any embodiment described in this disclosure, in any order and with any degree of repetition. For instance, processor 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Processor 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, apparatus 100 includes at least a at least a catheter 112 configured for intracardiac use, the at least a at least a catheter 112 comprising at least a transducer configured to detect cardiac phenomenon 116 and output at least a potential signal, as a function of cardiac phenomenon 116. As used in this disclosure, a “catheter” is a flexible tube inserted into the body to perform various medical procedures. In a non-limiting example, at least a catheter 112 may record and map at least a beat of a cardiac phenomenon 116 and output at least a visual element. In a non-limiting example, at least a catheter 112 may be used to facilitate the detection and mapping of cardiac activity, providing essential data for apparatus 100 to process and analyze. In a non-limiting example, at least a catheter 112 may be used in procedures such as cardiac ablation or electrophysiological studies to gather detailed information about heart rhythms. As used in this disclosure, a “cardiac phenomenon” is any physiological or pathological event, activity, or condition related to the function or behavior of the heart that can be detected or measured. Cardiac phenomenon 116 includes but is not limited to electrical signals, mechanical movements, pressure changes, and biochemical processes occurring within the heart or its surrounding tissues. Cardiac phenomenon 116 are crucial indicators of heart health and function, and they provide valuable data for diagnosing, monitoring, and treating various cardiac conditions. In a non-limiting example, a cardiac phenomenon 116 may refer to the electrical activity associated with the heart's rhythm, such as the depolarization and repolarization of cardiac cells that create the P wave, QRS complex, and T wave observed in an electrocardiogram (ECG). Transducer 120 in at least a catheter 112 may detect these electrical signals and output potential signals corresponding to these cardiac events, enabling real-time monitoring of heart rhythms and the identification of arrhythmias or other electrical abnormalities.

As used in this disclosure, a “transducer” is a device designed to convert one form of energy into another. In a non-limiting example, transducer 120 may facilitate the measurement, monitoring, and control of various physical quantities. Without limitation, this energy conversion capability may enable transducers to be used for various applications. For instance, without limitation, transducers may be used in ultrasound equipment to transform electrical energy into sound waves and vice versa, creating images of internal body structures. When a transducer converts a quantity of energy to an electrical voltage or an electrical current it is called a sensor. A measurable quantity of energy may include sound pressure, optical intensity, magnetic field intensity, thermal pressure, etc. When a transducer converts an electrical signal into another form of energy such as sound, light, mechanical movement, it is called an actuator. It should be noted that sound is incidentally a pressure field. Actuators allow the use of feedback at the source of the measurements. In a non-limiting embodiment, a transducer may detect at least a cardiac phenomenon 116 and output potential signal. In another non-limiting example, a transducer may include a plurality of clinical transducers. As used in this disclosure, a “plurality of clinical transducers” is a transducer device used in the medical field to measure, analyze, and/or quantify electrical signals in a body. As used in this disclosure, a “potential signal” is the electrical signal generated and output by a transducer in response to detecting cardiac phenomenon 116 within the heart. The potential signal may be indicative of the heart's electrical activity, which may be used for diagnostic or monitoring purposes. The potential signal may represent variations in electrical potential that occur as the heart undergoes its rhythmic contractions and relaxations, providing valuable data on the cardiac cycle and function. In a non-limiting example, the potential signal may be generated by a transducer embedded in at least a catheter 112 during an electrophysiological study. When at least a catheter 112 is positioned intracardially, transducer 120 may detect electrical impulses corresponding to the depolarization and repolarization phases of the cardiac cycle. The output potential signal may then be transmitted to an external monitoring system where it is displayed as an electrocardiogram (ECG) tracing. Continuing, the tracing may allow cardiologists to analyze the electrical activity and identify abnormalities such as arrhythmias or conduction blockages. In another non-limiting example, the potential signal may be used in a real-time cardiac monitoring system during a surgical procedure. Continuing, as at least a catheter 112 transducer may detect changes in intracardiac electrical activity, the potential signal is continuously sent to a monitoring device. Without limitation, surgeons may use this real-time data to make informed decisions about interventions, ensuring that the heart remains stable and functions properly throughout given procedure. Without limitation, the aforementioned application highlights the versatility of potential signals in providing critical, time-sensitive information in various medical contexts.

In another non-limiting example, cardiac phenomenon 116 may include the mechanical contraction and relaxation of the heart muscle, such as the pressure changes during the systolic and diastolic phases of the cardiac cycle. Transducer 120 may detect variations in intracardiac pressure and generate signals that reflect these pressure changes. This data may be used to assess cardiac output, diagnose conditions like heart failure or valvular heart disease, and guide therapeutic interventions by providing detailed insights into the heart's mechanical function.

This may include, without limitation, various types of signal data, such as analog signals, digital signals, time-series signal data, spatial signals, frequency signals, multi-dimensional signals, and the like. In a non-limiting example, an analog signal is any continuous-time signal representing some other quantity, i.e., analogous to another quantity. For example, and without limitation, in an analog audio signal, the instantaneous signal voltage varies continuously with the pressure of the sound waves. Typically, analog signal refers to electrical signals; however, mechanical, pneumatic, hydraulic, and other systems may also convey or be considered analog signals. In another non-limiting example, a digital signal is a signal that represents data as a sequence of discrete values; at any given time it can only take on, at most, one of a finite number of values. In some cases, digital signals may represent information in discrete bands of analog levels, wherein all levels within a band of values represent the same information state. In a non-limiting example, a digital signal may be represented as a digital circuit. Typically, digital circuit signals can have two possible valid values; a binary signal or logic signal wherein the binary signal and the logic signal are represented by two voltage bands: one voltage band that is near a reference value, and the other voltage value that is near the supply voltage. The voltage bands correspond to the two values “zero” and “one” (or “false” and “true”) of the Boolean domain, wherein at any given time, a binary signal represents one binary digit (bit). Without limitation, digital signals are generally used for communications and processing within electronic devices and computer systems. In another non-limiting example, time-series signal data is information in the form of a signal that is collected and recorded over consistent intervals of time. Without limitation, time-series signal data may be used in order to extract meaningful statistics and other characteristics of the data. Time-series signal data can be classified into two main types: continuous-time series signals and discrete-time signals. Continuous-time signals are signals that are measured and recorded over a continuous range, including, but not limited to, analog signals, such as sound waves and temperature measurements (from analog devices like analog thermometers). On the other hand, discrete-time signals are recorded at specific, distinct points. For example, and without limitation, discrete-time signals may include digital sensor measurements and financial market data sampled at fixed intervals. In another non-limiting example, potential signal 124 may include an electrocardiogram signal wherein the electrocardiogram signal may include an electrocardiogram datum. As used herein, an “electrocardiogram datum” is a single data point obtained from the electrical activity of the heart of a patient. An electrocardiogram datum may be derived from an electrocardiogram signal. In some embodiments, an electrocardiogram datum may include a rhythm strip electrocardiogram datum. As used herein, a “rhythm strip electrocardiogram datum” is a datum describing electrical activity detected using a single electrode. In some embodiments, an electrocardiogram datum may include a median beat electrocardiogram datum. As used herein, a “median beat electrocardiogram datum” is a datum describing electrical activity detected using a plurality of leads and/or electrodes. In some embodiments, an electrocardiogram datum may include data collected by 2, 3, 4, 5, 6, 7, 8, 9, 10, 11, 12, or more electrocardiogram leads. For example, an electrocardiogram datum may include a median beat collected by 12 electrocardiogram leads. A “lead,” as used in this disclosure, is one or more electrodes attached to the skin to detect a heart's electric signals. As used in this disclosure, a “standard 12-lead electrocardiogram signal” is a measurement the electrical activity of a heart from 12 different perspectives. In a non-limiting embodiment a standard 12-lead electrocardiogram signal may include a graphical record of the direction and magnitude of the electrical activity generated by the depolarization and repolarization of the atria and ventricles of the heart. As used in this disclosure, an “electroanatomic map” is a detailed, three-dimensional representation of the electrical activity and anatomical structure of the heart. In a non-limiting example, the electroanatomic map may be created using data collected from a catheter that records and maps cardiac phenomena. In another non-limiting example, the electroanatomic map mya provide a visual depiction of the heart's electrical impulses and physical form, enabling precise identification and analysis of areas that may be causing abnormal heart rhythms or other cardiac issues. The electroanatomic map integrates both the electrical signals and the spatial geometry of the heart, offering a comprehensive tool for diagnosis and treatment planning. In a non-limiting example, an electroanatomic map may be created during an electrophysiological study where a catheter is navigated through the heart to record electrical activity. The data collected from various points within the heart is used to construct a three-dimensional map that highlights regions of interest, such as areas with abnormal electrical pathways or scar tissue. This map can be displayed on a monitor, providing clinicians with a visual guide to target specific areas for ablation therapy, thereby improving the precision and effectiveness of the treatment. In another non-limiting example, the electroanatomic map may be employed during a cardiac procedure to continuously update the map in real-time as at least a catheter 112 moves within the heart. This dynamic mapping allows for immediate adjustments based on the current electrical activity and anatomical changes observed during the procedure. Such real-time updates may be particularly useful in complex cases where the anatomy and electrical activity of the heart vary significantly from patient to patient, ensuring that the intervention is tailored to the individual's specific cardiac structure and function.

Still referring to FIG. 1, apparatus 100 includes at least a localization system 128 configured to detect at least a position signal 132 as a function of a location of the at least a catheter. As used in this disclosure, a “localization system” is a specialized apparatus designed to detect and determine the position of a catheter within a body or environment by utilizing position signal 132. These signals are a function of at least a catheter 112 location, enabling precise tracking and navigation during medical procedures. In a non-limiting example, the purpose of at least a localization system 128 is to enhance the safety and efficacy of catheter-based interventions by providing critical spatial information. As used in this disclosure, a “position signal” is a signal generated by localization system 128 to determine the location of a catheter within the body. With continued reference to FIG. 1, in a non-limiting example, localization system 128 may be consistent with one or more aspects of the localization system described in attorney docket number 1518-160USU1, U.S. patent application Ser. No. 18/764,853, filed on Jul. 5, 2024, titled “SYSTEM AND METHOD FOR LOCATING A MEDICAL DEVICE USING AN ELECTRICAL FIELD CREATION,” which is incorporated by reference herein in its entirety.

With continued reference to FIG. 1, at least a localization system 128 may include one or more of an electromagnetic localization system, an ultrasound-based localization system, an optical localization system, and an impedance-based localization system. As used in this disclosure, an “electromagnetic localization system” is a type of localization technology that uses electromagnetic fields to determine the precise position and orientation of objects within a given space. This system typically involves generating a low-frequency electromagnetic field in the area of interest, and then tracking the position of sensors or coils that respond to this field. The sensors may be integrated into catheters or other medical instruments, allowing for accurate real-time tracking of their location and movement within the body. In the context of electroanatomic mapping, the electromagnetic localization system enables the precise localization of at least a catheter 112 tip within the heart. This is achieved by placing electromagnetic field generators around the patient and using sensors on at least a catheter 112 to detect the field. The system calculates the exact position and orientation of at least a catheter 112 by measuring the electromagnetic field's strength and direction at the sensor's location. This information is then transmitted to the processor, which uses it to construct a detailed, three-dimensional map of the heart's anatomy. This technology is essential for guiding medical procedures such as catheter ablation, where precise navigation within the heart is critical. By providing accurate and real-time positional data, the electromagnetic localization system ensures that at least a catheter 112 can be maneuvered safely and effectively to target areas of abnormal electrical activity, thereby improving the outcomes of the procedure.

With continued reference to FIG. 1, as used in this disclosure, an “ultrasound-based localization system” is a method used to determine the position and movement of objects within the body by employing high-frequency sound waves. The ultrasound-based localization system may involve the use of an ultrasound transducer that emits sound waves, which then reflect off internal structures and are captured by transducer 120 or other sensors. Continuing, the reflected sound waves are processed to create real-time images or data points that represent the location and motion of the tracked object, such as a catheter or other medical instruments. The ultrasound-based localization system may be particularly useful in medical procedures because it provides real-time, non-invasive visualization of internal body structures. The ultrasound-based localization system may allow clinicians to guide instruments accurately within the body, enhancing the precision and safety of procedures like catheter ablation, biopsies, or other interventions. This technology is often integrated with other systems to provide comprehensive spatial and functional mapping of the area being treated. For example, at least a localization system 128 may utilize ultrasound technology, where an array of ultrasound transducers is positioned around the patient. At least a catheter 112, may be fitted with miniature ultrasound receivers, detects the emitted ultrasound waves. At least a localization system 128 may calculate at least a catheter 112 position based on the time it takes for the ultrasound waves to reach the receivers, allowing for precise localization of at least a catheter 112 tip during a procedure.

With continued reference to FIG. 1, as used in this disclosure, “optical localization system” is a method of determining the position and movement of objects using light, typically through the use of cameras and other optical sensors. Optical localization system technology may capture visual data from the tracked object and processes this information to calculate its precise location and trajectory in real-time. In an optical localization system system, reflective markers or LED lights may be attached to the object being tracked, such as a catheter tip. Cameras positioned around the area capture the light reflected or emitted by these markers, and software algorithms analyze the captured images to triangulate the exact position of the markers. This data is then transmitted to the processor, which integrates it with other signals to create a comprehensive map of the object's movement within the heart. This method is highly accurate and provides detailed spatial information, making it particularly useful in medical applications where precise positioning is crucial. Optical localization system can be used in conjunction with other localization methods to enhance the overall accuracy and reliability of the electroanatomic mapping system.

With continued reference to FIG. 1, as used in this disclosure, “impedance-based localization system” is a technique used to determine the position of a catheter or other medical device within the body by measuring the electrical impedance between the device and electrodes placed on the patient's body. This method involves passing a small, alternating current through the body and measuring the resulting voltage at different points, allowing the system to calculate the impedance. At least a localization system 128 can then use these impedance measurements to triangulate the exact position of at least a catheter 112 tip within the heart or other body cavities. Impedance varies with the distance and the type of tissue between at least a catheter 112 and the electrodes, enabling precise tracking of the device's location. This technique is particularly useful in electroanatomic mapping and other procedures requiring accurate real-time positioning of medical instruments within the body. In a non-limiting example, position signal 132 may be generated using electromagnetic fields, ultrasound, or other tracking technologies to provide real-time spatial information about at least a at least a catheter 112 position. In a non-limiting example, apparatus 100 may employ other tracking technologies, such as optical localization system or impedance-based localization, to generate position signal 132. Optical localization system uses cameras and reflective markers on at least a catheter 112 to capture its movement and position, while impedance-based localization measures electrical impedance differences between at least a catheter 112 and the body tissues. These methods provide accurate real-time spatial information that processor 104 uses alongside the potential signal 124.

Still referring to FIG. 1, processor 104 is configured to receive at least a potential signal 124 from the at least a transducer 120 and the at least a position signal from at least a localization system 128. In a non-limiting example, processor 104 may utilize an electrocardiogram (ECG) transducer to capture electrical signals from the heart, which are indicative of the cardiac cycle. The ECG transducer may detect the electrical potentials generated by the heart's activity and transmits these signals to processor 104. Processor 104 may then analyze the amplitude and frequency of these signals to identify specific heartbeats, allowing for precise timing and coordination with the electroanatomic mapping system. In a non-limiting example, at least a localization system 128 may use a magnetic or electromagnetic field to determine the exact position of a catheter tip within the heart. At least a localization system 128 may generate position signals wherein processor 104 may be configured to receive position signal 132. Continuing, position signal 132 may be used to create a three-dimensional map of the heart's anatomy, showing the real-time position of at least a catheter 112. Without limitation, the position data with potential signal 124 may be combined and processor 104 may map the electrical activity across different regions of the heart, enhancing the accuracy of the electroanatomic mapping. In a non-limiting example, processor 104 may employ advanced algorithms to filter and interpret potential signal 124 received from multiple transducers placed at various points on the patient's body. Continuing, the algorithms may differentiate between true cardiac signals and noise or artifacts, ensuring that only relevant electrical activity is used for mapping. Processor 104 may integrate the filtered data with the positional information from localization system. In a non-limiting example, this integration may allow clinicians to visualize the propagation of electrical impulses through the heart's chambers and identify areas of abnormal conduction.

With continued reference to FIG. 1, at least a potential signal 124 may include a plurality of electrocardiogram data. As used in the current disclosure, a “electrocardiogram data” is a signal representative of electrical activity of heart. The electrocardiogram data may consist of several distinct waves and intervals, each representing a different phase of the cardiac cycle. These waves may include the P-wave, QRS complex, T wave, U wave, and the like. The P-wave may represent atrial depolarization (contraction) as the electrical impulse spreads through the atria. The QRS complex may represent ventricular depolarization (contraction) as the electrical impulse spreads through the ventricles. The QRS complex may include three waves: Q wave, R wave, and S wave. The T-wave may represent ventricular repolarization (recovery) as the ventricles prepare for the next contraction. The U-wave may sometimes be present after the T wave, it represents repolarization of the Purkinje fibers. The intervals between these waves may provide information about the duration and regularity of various phases of the cardiac cycle.

Still referring to FIG. 1, processor 104 determines, using beat detection algorithm 136, at least a beat template 140 as a function of at least a potential signal 124. As used in this disclosure, a “beat detection algorithm” is a computational method employed by a processor to identify and isolate the characteristic beats or pulses within a signal, particularly from biological sources such as the heart. This algorithm filters out extraneous noise and interference from the input signal to accurately detect the occurrence and timing of beats, which is essential for various diagnostic and monitoring applications. Beat detection algorithm 136 may involve signal processing techniques such as thresholding, pattern recognition, and frequency analysis to distinguish the relevant heartbeat signals from the background noise. In a non-limiting example, beat detection algorithm 136 may be used in an electrocardiogram (ECG) monitoring system. Processor 104, utilizing beat detection algorithm 136, may filter out muscle noise, electrical interference, and other artifacts from the raw ECG signal to accurately detect the QRS complex, which represents the main spike in the heartbeat cycle. Apparatus 100 may provide reliable data on heart rate and rhythm, which are critical for diagnosing arrhythmias and other cardiac conditions. As used in this disclosure, a “beat template” is a predefined pattern or set of characteristics representing a typical heartbeat derived from the analysis of electrical signals detected by the system. Without limitation beat template may include single lead templates and multi lead templates. In a non-limiting example, a single lead template may be derived from the electrical signals detected by a single lead of an electrocardiogram (ECG), continuing, this template may capture the characteristic features of a typical heartbeat, such as the shape, duration, and amplitude of the P wave, QRS complex, and T wave, as observed from that specific lead. The single lead template may be used by beat detection algorithm 136 to identify and compare individual heartbeats detected by the same lead during electroanatomic mapping. This approach ensures that the detected beats are accurately represented in both the geometric and electrical aspects of the map, providing a reliable reference for identifying deviations or abnormalities in the heart's electrical activity. In another non-limiting example, a multi-lead template may be created by graphing the electrical signals from multiple leads of an ECG. This template may capture the common features of a typical heartbeat as observed from various perspectives around the heart, providing a more comprehensive representation of the cardiac cycle. Without limitation, the multi-lead template may be used by beat detection algorithm 136 to identify and compare individual heartbeats detected by multiple leads during electroanatomic mapping. Without limitation, the multi-lead template may enhance the accuracy and reliability of beat detection, allowing for a more detailed analysis of the heart's electrical activity.

In another non-limiting example, the template matching may be performed using QRS or P waves in Sinus Rhythm. Without limitation, the template matching may be performed using QRS or P waves in discontinuous rhythms, wherein multi template matching may be required. In a non-limiting example, beat template 140 may be created by processor 104 using a beat detection algorithm that processes potential signal 124 received from the heart. In another non-limiting example, beat template 140 may serve as a reference for identifying and comparing individual heartbeats during electroanatomic mapping, allowing the system to accurately detect and map the electrical activity of the heart. In a non-limiting example, processor 104 may generate a beat template by averaging the electrical signals from multiple detected heartbeats over a specific period. This averaged template may capture the common features of a typical heartbeat, such as the shape, duration, and amplitude of the P wave, QRS complex, and T wave. Processor 104 may then use this beat template to identify deviations in subsequent heartbeats, helping to detect arrhythmias or other cardiac abnormalities. In a non-limiting example, processor 104 might create multiple beat templates to account for different types of heartbeats, such as normal beats, premature ventricular contractions, or atrial fibrillations. Each template may be tailored to the unique characteristics of these heartbeat types. By comparing incoming potential signal 124 to these templates, processor 104 may classify the heartbeats and provide detailed diagnostic information to clinicians, enabling more precise treatment planning and intervention.

Still referring to FIG. 1, processor 104 compares, using beat detection algorithm 136, the at least a beat template with at least a potential signal 124 over time. Without limitation, beat detection algorithm 136 may compare single lead templates and multi-lead templates. In a non-limiting example, processor 104 may compare, using beat detection algorithm 136, beat template 140 with potential signal 124 over time by continuously analyzing the incoming data from transducer 120 and matching it against the predefined characteristics of beat template 140. In another non-limiting example, processor 104 may compare, using beat detection algorithm 136, beat template 140 with potential signal 124 over time by applying a cross-correlation technique to identify the degree of similarity between the real-time signal and beat template 140. In a non-limiting example, the cross-correlation technique involves shifting beat template 140 across potential signal 124 and calculating the correlation coefficient at each position. This coefficient measures the similarity between beat template 140 and the segment of potential signal 124 at that position. The position with the highest correlation coefficient indicates the best match between beat template 140 and potential signal 124. In a non-limiting example, processor 104 uses this information to accurately detect the timing and occurrence of beats within potential signal 124. In a non-limiting example, processor 104 compares, using beat detection algorithm 136, beat template 140 with potential signal 124 over time by calculating the root mean square (RMS) error between the detected signal and beat template 140 to determine the accuracy of the match. In a non-limiting example, the RMS error calculation involves taking the difference between the amplitude of beat template 140 and the corresponding segment of potential signal 124 at each time point. These differences are then squared to eliminate negative values and emphasize larger discrepancies. The squared differences are averaged over the entire comparison window, and the square root of this average is taken to obtain the RMS error. A lower RMS error indicates a higher degree of similarity between beat template 140 and potential signal 124, signifying a more accurate match. In a non-limiting example, processor 104 uses the RMS error to continuously adjust and refine the beat detection process, ensuring precise identification of cardiac events. In a non-limiting example, processor 104 compares, using beat detection algorithm 136, beat template 140 with potential signal 124 over time by employing a pattern recognition algorithm that identifies key features of beat template 140 within potential signal 124. In a non-limiting example, the pattern recognition algorithm begins by extracting distinctive features from beat template 140, such as the amplitude, duration, and shape of the P wave, QRS complex, and T wave. These features are encoded into a feature vector that represents beat template 140. In a non-limiting example, processor 104 continuously analyzes potential signal 124 to extract similar feature vectors from the incoming data. The pattern recognition algorithm then compares these feature vectors to the feature vector of beat template 140 using similarity measures such as Euclidean distance or cosine similarity. The algorithm identifies segments of potential signal 124 that closely match beat template 140 based on these similarity measures. In a non-limiting example, once a match is identified, processor 104 marks the timing and occurrence of the detected beat within potential signal 124. This information is then used to update the electroanatomic map, ensuring that the detected beats are accurately represented in both the geometric and electrical aspects of the map. The continuous feedback provided by the pattern recognition algorithm allows for real-time adjustments and refinements, improving the overall accuracy and reliability of the beat detection process.

Still referring to FIG. 1, processor 104 detects, using beat detection algorithm 136, a beat timing as a function of the comparison of the at least a beat template and the at least a potential signal. As used in this disclosure, “beat timing” is the precise moment or interval at which a cardiac event, such as a heartbeat, occurs within a cardiac cycle. Without limitation, beat timing 144 may be determined by looking at the initial index of the best matched segment after performing template matching in real-time. In a non-limiting example, beat timing 144 may be determined after the match is found. In a non-limiting example, beat timing 144 may be determined by identifying the peak of the QRS complex in potential signal 124 and aligning it with the corresponding peak in beat template 140. In a non-limiting example, beat timing 144 may be calculated by measuring the interval between successive R waves in potential signal 124 and comparing it to the interval in beat template 140. In a non-limiting example, beat timing 144 may be assessed by calculating the time difference between the onset of the P wave in potential signal 124 and the onset of the P wave in beat template 140. In a non-limiting example, processor 104 may detect, using beat detection algorithm 136, beat timing 144 as a function of the comparison of beat template 140 and potential signal 124 by identifying the peak of the QRS complex in potential signal 124 and aligning it with the corresponding peak in beat template 140. In a non-limiting example, processor 104 may detect, using beat detection algorithm 136, beat timing 144 as a function of the comparison of beat template 140 and potential signal 124 by calculating the time difference between the onset of the P wave in potential signal 124 and the onset of the P wave in beat template 140. In a non-limiting example, processor 104 may detect, using beat detection algorithm 136, beat timing as a function of the comparison of beat template 140 and potential signal 124 by measuring the interval between successive R waves in potential signal 124 and comparing it to the interval in beat template 140 to determine the timing of each beat.

With continued reference to FIG. 1, apparatus 100 may be configured to allow a user to select which body surface leads to use for beat detection algorithm 136. Without limitation, once at least a lead is selected by the user, the derivative function is calculated for the selected at least a lead. Continuing, beat detection algorithm 136 may take a sample by sample comparison between the derivative graphs of the selected at least a lead against the derivative graph of the incoming at least a potential signal 124. For instance, without limitation, a clinician may choose leads V1, V2, and V3 for analysis, and the derivative function for these selected leads may be calculated. Continuing, beat detection algorithm 136 may take a sample-by-sample comparison between the derivative graphs of the selected leads against the derivative graph of the incoming potential signal 124, ensuring accurate beat detection and alignment.

With continued reference to FIG. 1, wherein beat detection algorithm 136 is further configured to calculate a derivative of potential signal 124 for each lead, sum the derivative of potential signal for each lead, identify a local minimum derivative value of the summed potential signal 124, and compare, using the local minimum derivative value, the summed potential signal with the at least a beat template. As used in this disclosure, a “derivative of the potential signal” is the rate of change of potential signal 124 with respect to time. In a non-limiting example, calculating the derivative of potential signal 124 helps to highlight rapid changes in the signal, such as the steep slopes of the QRS complex, making it easier to detect and align cardiac events accurately. In a non-limiting example, identifying a local minimum derivative value of the summed potential signal involves analyzing the summed derivative signal to find points where the rate of change transitions from negative to positive. This may indicate a local minimum. The algorithm may scan through the summed potential signal, comparing each point to its neighbors to determine if it is a local minimum. If a point has a lower value than the points immediately before and after it, it is identified as a local minimum. This local minimum derivative value may then used to align beat template 140 with potential signal 124 for accurate beat detection. Without limitation, the local minimum derivative value may only include a single point. For example, without limitation, the local minimum derivative value may not be calculated for the entire graph of potential signal 124.

With continued reference to FIG. 1, calculating a derivative of potential signal 124 for each lead, summing the derivative of potential signal for each lead, identifying a local minimum derivative value of the summed potential signal 124, and comparing the summed potential signal with the at least a beat template may help identify specific features within cardiac phenomenon 116, such as different types of heartbeats. Without limitation, beat detection algorithm 136, as described, may analyze potential signal 124 collected from multiple leads in an electrocardiogram (ECG) for identify and classify different types of heartbeats based on their unique waveform characteristics. The comparison process involves “matching” the summed potential signal with these templates. Mathematically, this may be achieved through various methods such as cross-correlation, where beat detection algorithm 136 slides each template over the summed signal and calculates a correlation coefficient at each position. Continuing, the position where the highest correlation occurs may indicate the best match. Continuing, computing system may determine how well the pattern of the summed signal aligns with each template.

With continued reference to FIG. 1, wherein apparatus 100 may include a potential signal filter and a motion filter. As used in this disclosure, a “potential signal filter” is a component or algorithm designed to remove unwanted noise and artifacts from potential signal 124 to improve the accuracy of beat detection. In a non-limiting example, a potential signal filter may include a high-pass filter to eliminate low-frequency noise and a low-pass filter to remove high-frequency noise. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units. In a non-limiting example, the potential signal filter may also include notch filters to specifically target and remove powerline interference. In a non-limiting example, the potential signal filter may be configured to automatically detect and notify the user of leads with excessive noise, allowing for real-time adjustments and ensuring the integrity of the potential signal used for beat detection. A “high frequency noise” refers to unwanted disturbances or variations in the signal that occur at high frequencies. As used in this disclosure, a “powerline noise” is a type of noise that originates from the electrical power grid. Without limitation, the powerline noise may manifest as a sinusoidal interference at the frequency of the power supply. Without limitation the automatic filtering to remove high frequency and powerline noises may improve the signal to noise ratio (SNR). The automatic filtering may include detection of leads with excessive noise and/or providing the user with a notification that asks whether the system should fix or not use the signal with excessive noise.

With continued reference to FIG. 1, as used in this disclosure, a “motion filter” is a component or algorithm designed to compensate for motion artifacts in position signal 132, which may arise from cardiac or respiratory movements. Without limitation, the motion filter may compensate for cardiac motion. Continuing, the motion compensation may be performed on the time aligned positional signal data, such as at least a position signal 132, that is received from the at least a localization system 128. In a non-limiting example, the motion filter may use algorithms to detect and correct for shifts in the signal caused by the physical movement of the heart or the patient's breathing. In a non-limiting example, the motion filter may employ techniques such as signal averaging, adaptive filtering, or motion tracking to isolate and remove motion-related noise, ensuring that the potential signal accurately reflects the underlying cardiac activity.

With continued reference to FIG. 1, in a non-limiting example, the motion filter may be consistent with one or more aspects of the motion filter described in attorney docket number 1518-149USU1, U.S. patent application Ser. No. 18/788,704, filed on Jul. 30, 2024, titled “APPARATUS AND METHOD FOR DETERMINING THE QUALITY OF A POTENTIAL SIGNAL USING CATHETER VELOCITY DURING ELECTRO-ANATOMICAL MAPPING,” which is incorporated by reference herein in its entirety.

With continued reference to FIG. 1, wherein the potential signal filter may be configured to detect at least a noise in the at least a potential signal and remove the at least a noise from the at least a potential signal. As used in this disclosure, “at least a noise” is any unwanted disturbance or interference within the potential signal that can affect the accuracy of beat detection. In a non-limiting example, noise may include high-frequency noise, powerline interference, or baseline wander, which can obscure the true cardiac signals and lead to inaccurate mapping. In a non-limiting example, high-frequency noise may originate from electronic devices or muscle contractions. In a non-limiting example, powerline interference May manifest as a 50/60 Hz hum from electrical power sources. In a non-limiting example, the potential signal filter may be used to automatically filter noise during template matching.

With continued reference to FIG. 1, the motion filter may be configured to detect a cardiac motion and a respiratory motion and remove the cardiac motion and respiratory motion from the at least a position signal. As used in this disclosure, “cardiac motion” is the physical movement of the heart during its contraction and relaxation cycles. In a non-limiting example, cardiac motion may include the mechanical activities associated with the systolic and diastolic phases of the cardiac cycle, such as the ejection of blood from the ventricles and the filling of the atria. In a non-limiting example, cardiac motion may cause motion artifacts in the potential signal, which can affect the accuracy of beat detection and mapping. In a non-limiting example, a motion filter may be used to detect and compensate for these motion artifacts, ensuring that the potential signal accurately reflects the underlying cardiac activity. As used in this disclosure, “respiratory motion” is the physical movement of the chest and abdomen during the process of breathing, which can cause displacement of internal organs, including the heart. In a non-limiting example, respiratory motion may include the expansion and contraction of the lungs during inhalation and exhalation, which can lead to shifts in the position of the heart and other thoracic structures. In a non-limiting example, respiratory motion may cause motion artifacts in the potential signal, affecting the accuracy of beat detection and mapping. In a non-limiting example, the motion filter may be used to detect and compensate for these motion artifacts, ensuring that the potential signal accurately reflects the underlying cardiac activity. In a non-limiting example, cardiac motion and respiratory motion may include baseline wander, which can obscure the true cardiac signals and lead to inaccurate mapping. In a non-limiting example, baseline wander may result from patient movement or respiration, causing slow drifts in the signal baseline.

With continued reference to FIG. 1, processor 104 may provide continuous feedback to a user through a display device, using a notification system, wherein the continuous feedback comprises information associated with signal detection and beat morphology stability. As used in this disclosure, “continuous feedback” is the ongoing, real-time provision of information to a user about the status or performance of a system or process. In a non-limiting example, continuous feedback may include updates on signal detection and beat morphology stability during an electroanatomic mapping procedure. In a non-limiting example, continuous feedback may be provided through a notification system that alerts the user to any issues or changes in the detected signals, allowing for immediate adjustments and ensuring the accuracy and reliability of the mapping process. As used in this disclosure, a “notification system” is a component or set of components designed to alert the user to specific events, statuses, or changes within a system or process. In a non-limiting example, a notification system may provide continuous feedback by alerting the user to issues or changes in detected signals during an electroanatomic mapping procedure. In a non-limiting example, the notification system may include visual alerts, such as pop-up messages or color-coded indicators on a display device, as well as auditory alerts, such as beeps or spoken messages, to ensure the user is promptly informed of any significant events or required adjustments. In a non-limiting example, the notification system may be integrated with the user interface to allow for immediate user interaction and response to the alerts, ensuring the accuracy and reliability of the mapping process. As used in this disclosure, “beat morphology stability” is the consistency of the shape and structure of detected heartbeats over time. In a non-limiting example, beat morphology stability may be assessed by comparing the waveform characteristics of consecutive beats, such as the amplitude, duration, and shape of the P wave, QRS complex, and T wave. In a non-limiting example, stable beat morphology indicates that the detected beats are consistent and reliable, which is crucial for accurate electroanatomic mapping. In a non-limiting example, continuous feedback on beat morphology stability may be provided to the user to ensure that beat detection algorithm 136 is functioning correctly and that the detected beats are suitable for mapping and analysis. As used in this disclosure, a “display device” refers to an electronic device that visually presents information to the entity. In some cases, display device may be configured to project or show visual content generated by computers, video devices, or other electronic mechanisms. In some cases, display device may include a liquid crystal display (LCD), a cathode ray tube (CRT), a plasma display, a light emitting diode (LED) display, and any combinations thereof. In a non-limiting example, one or more display devices may vary in size, resolution, technology, and functionality. Display device may be able to show any data elements and/or visual elements as listed above in various formats such as, textural, graphical, video among others, in either monochrome or color. Display device may include, but is not limited to, a smartphone, tablet, laptop, monitor, tablet, and the like. Display device may include a separate device that includes a transparent screen configured to display computer generated images and/or information. In some cases, display device may be configured to present a graphical user interface (GUI) to a user, wherein a user may interact with a GUI. In some cases, a user may view a GUI through display. Additionally, or alternatively, processor 104 be connected to display device. In one or more embodiments, transmitting the continuous feedback may include displaying the continuous feedback at display device using a visual interface. As used in this disclosure, a “visual interface” is a digital display that presents information, options, interactive elements to users in an intuitive and visually appealing manner. In some embodiments, visual interface may include at least an interface element. As used in this disclosure, “at least an interface element” is a portion of visual interface. In a non-limiting example, at least an interface element may include, without limitation, a button, a link, a checkbox, a text entry box and/or window, a drop-down list, a slider, or any other interface element that may occur to a person skilled in the art upon reviewing the entirety of this disclosure. In some embodiments, at least an interface element may include an event handler. As used in this disclosure, an “event handler” is a software routine or function designed to process specific events or actions within a system or application. In a non-limiting example, an event handler may be triggered by user interactions such as clicks, key presses, or mouse movements, and it executes predefined actions in response to these events. In a non-limiting example, an event handler may be used in a graphical user interface (GUI) to manage user inputs and provide real-time feedback, ensuring a seamless and interactive user experience. In a non-limiting example, an event handler may also be employed to handle system-generated events, such as timers or network messages, to perform tasks like updating data displays or processing incoming information.

With continued reference to FIG. 1, beat detection algorithm 136 may be configured to match a first template of the at least a beat template and a second template of the at least a beat template with the at least a potential signal as a function of the beat timing. As used in this disclosure, a “first template” is a predefined pattern or set of characteristics representing a specific type of heartbeat or cardiac event used as a reference for comparison with incoming potential signals. In a non-limiting example, the first template may be created by averaging the electrical signals from multiple detected heartbeats over a specific period to capture the common features of a typical heartbeat. In a non-limiting example, the first template may include the shape, duration, and amplitude of the P wave, QRS complex, and T wave. In a non-limiting example, the first template is used by beat detection algorithm 136 to identify and compare individual heartbeats during electroanatomic mapping, ensuring accurate detection and mapping of the heart's electrical activity. As used in this disclosure, a “second template” is a predefined pattern or set of characteristics representing a different type of heartbeat or cardiac event used as a reference for comparison with incoming potential signals. In a non-limiting example, the second template may be created by averaging the electrical signals from multiple detected heartbeats of a different type over a specific period to capture the common features of that particular heartbeat. In a non-limiting example, the second template may include the shape, duration, and amplitude of the P wave, QRS complex, and T wave specific to that type of heartbeat. In a non-limiting example, the second template is used by beat detection algorithm 136 to identify and compare individual heartbeats during electroanatomic mapping, ensuring accurate detection and mapping of the heart's electrical activity for different types of beats.

With continued reference to FIG. 1, wherein matching multiple templates may include a P wave template, a QRS wave template, and premature ventricular contraction (PVC) wave template. Without limitation, the template matching may be performed on the electrical signal data from the at least a potential signal 124. As used in this disclosure, a “P wave template” is a predefined pattern representing the P wave in an electrocardiogram (ECG), which corresponds to the depolarization of the atria. In a non-limiting example, the P wave template includes the characteristic features of the P wave, such as its amplitude, duration, and shape. In a non-limiting example, the P wave template is used by beat detection algorithm 136 to identify and compare individual P waves during electroanatomic mapping, ensuring accurate detection and mapping of atrial activity. In a non-limiting example, the P wave template may be created by averaging the P waves from multiple detected heartbeats to capture the common features of atrial depolarization. As used in this disclosure, a “QRS wave template” is a predefined pattern representing the QRS complex in an electrocardiogram (ECG), which corresponds to the depolarization of the ventricles. In a non-limiting example, the QRS wave template includes the characteristic features of the QRS complex, such as its amplitude, duration, and shape. In a non-limiting example, the QRS wave template is used by beat detection algorithm 136 to identify and compare individual QRS complexes during electroanatomic mapping, ensuring accurate detection and mapping of ventricular activity. In a non-limiting example, the QRS wave template may be created by averaging the QRS complexes from multiple detected heartbeats to capture the common features of ventricular depolarization. As used in this disclosure, a “PVC wave template” is a predefined pattern representing the premature ventricular contraction (PVC) wave in an electrocardiogram (ECG), which corresponds to an early depolarization of the ventricles. In a non-limiting example, the PVC wave template includes the characteristic features of the PVC wave, such as its amplitude, duration, and shape. In a non-limiting example, the PVC wave template is used by beat detection algorithm 136 to identify and compare individual PVC waves during electroanatomic mapping, ensuring accurate detection and mapping of premature ventricular contractions. In a non-limiting example, the PVC wave template may be created by averaging the PVC waves from multiple detected heartbeats to capture the common features of premature ventricular depolarization. In a non-limiting example, beat detection algorithm 136 may use the aforementioned templates to identify and classify different types of heartbeats within the potential signal. Beat detection algorithm 136 continuously compares the incoming potential signal to each of the templates to find the best match. By matching the potential signal to the P wave template, the algorithm can identify atrial activity. By matching the potential signal to the QRS wave template, the algorithm can identify normal ventricular activity. By matching the potential signal to the PVC wave template, the algorithm can identify premature ventricular contractions. In a non-limiting example, the multi-template matching approach allows for a more comprehensive analysis of the cardiac cycle, enabling the detection of various types of heartbeats and arrhythmias. Continuing, processor 104 may use this information to update the electroanatomic map, providing a detailed representation of both the electrical and anatomical aspects of the heart.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

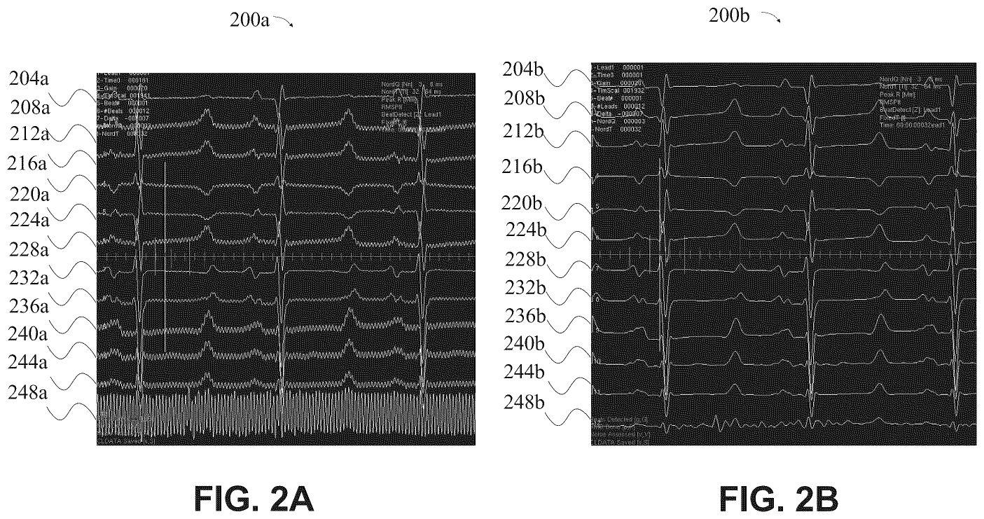

Referring now to FIG. 2A-B, where FIG. 2A is an illustration of a 12-lead potential signal graph of unacceptable noise, 200a and FIG. 2B, an illustration of a 12-lead potential signal graph of noise during an experiment, 200b. In FIG. 2A, the graph shows the raw data from a 12-lead ECG, wherein 204a represents lead 1, 208a represents lead 2, 212a represents lead 3, 216a represents lead 4, 220a represents lead 5, 224a represents lead 6, 228a represents lead 7, 232a represents lead 8, 236a represents lead 9, 240a represents lead 10, 244a represents lead 11, 248a represents lead 12, highlighting the presence of unacceptable noise levels that can interfere with accurate beat detection. The noise may originate from various sources, such as electronic devices, muscle contractions, or powerline interference, and is characterized by irregular, high-amplitude fluctuations that obscure the true cardiac signals. In a non-limiting example, the potential signal filter may be configured to detect and remove this noise to improve the signal-to-noise ratio (SNR) and ensure accurate beat detection. The filter may include high-pass and low-pass filters to eliminate low-frequency and high-frequency noise, respectively, as well as notch filters to target specific interference, such as powerline noise.

Referring to FIG. 2B, the graph illustrates the filtered data from the same 12-lead ECG during an experiment wherein 204b represents lead 1, 208b represents lead 2, 212b represents lead 3, 216b represents lead 4, 220b represents lead 5, 224b represents lead 6, 228b represents lead 7, 232b represents lead 8, 236b represents lead 9, 240b represents lead 10, 244b represents lead 11, 248b represents lead 12. The potential signal filter has been applied to remove the unacceptable noise, resulting in a cleaner signal with reduced amplitude fluctuations. This filtered signal allows for more accurate detection of cardiac events, such as the P wave, QRS complex, and T wave, and improves the overall reliability of the electroanatomic mapping process. In a non-limiting example, the beat detection algorithm may use the filtered potential signal to create beat templates and compare them with the incoming data to identify and classify different types of heartbeats. The continuous feedback provided by the notification system ensures that the user is informed of any issues or changes in the detected signals, allowing for immediate adjustments and maintaining the accuracy and reliability of the mapping process.

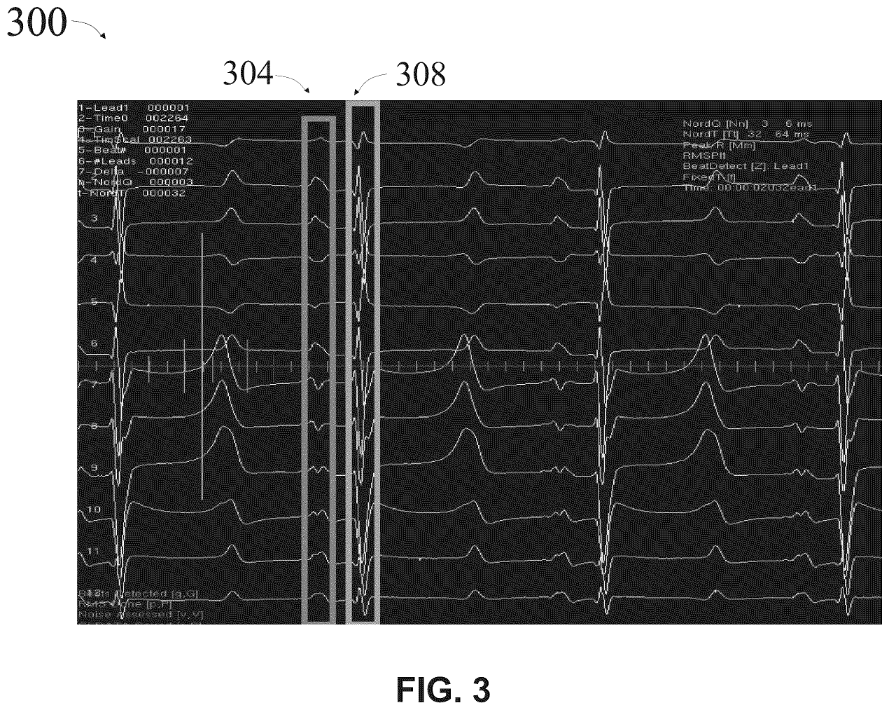

Referring now to FIG. 3, an illustration of a 12-lead potential signal graph wherein a window is selected for a P template and a QRS template, 300. The graph 300 shows the electrical activity of the heart as detected by a 12-lead electrocardiogram (ECG) system, highlighting specific segments used for template creation. In a non-limiting example, P template 304 may be a predefined pattern representing the P wave in the ECG signal. P template 304 may include the characteristic features of the P wave, such as the amplitude, duration, and shape. P template 304 may be used by the beat detection algorithm to identify and compare individual P waves during electroanatomic mapping, ensuring accurate detection and mapping of atrial activity. In a non-limiting example, QRS template 308 may be a predefined pattern representing the QRS complex in the ECG signal. QRS template 308 may include the characteristic features of the QRS complex, such as amplitude, duration, and shape. QRS template 308 may be used by the beat detection algorithm to identify and compare individual QRS complexes during electroanatomic mapping, ensuring accurate detection and mapping of ventricular activity. In a non-limiting example, the selection of P template 304 and QRS template 308 may be performed using a liberal onset to offset approach, which allows for a broad window of the ECG signal to be captured. This selection may be made using a mouse or trackpad, providing flexibility and ease of use for the operator. The onset points of the P wave and QRS complex may serve as the beat timing reference, ensuring precise alignment and accurate detection of cardiac events.

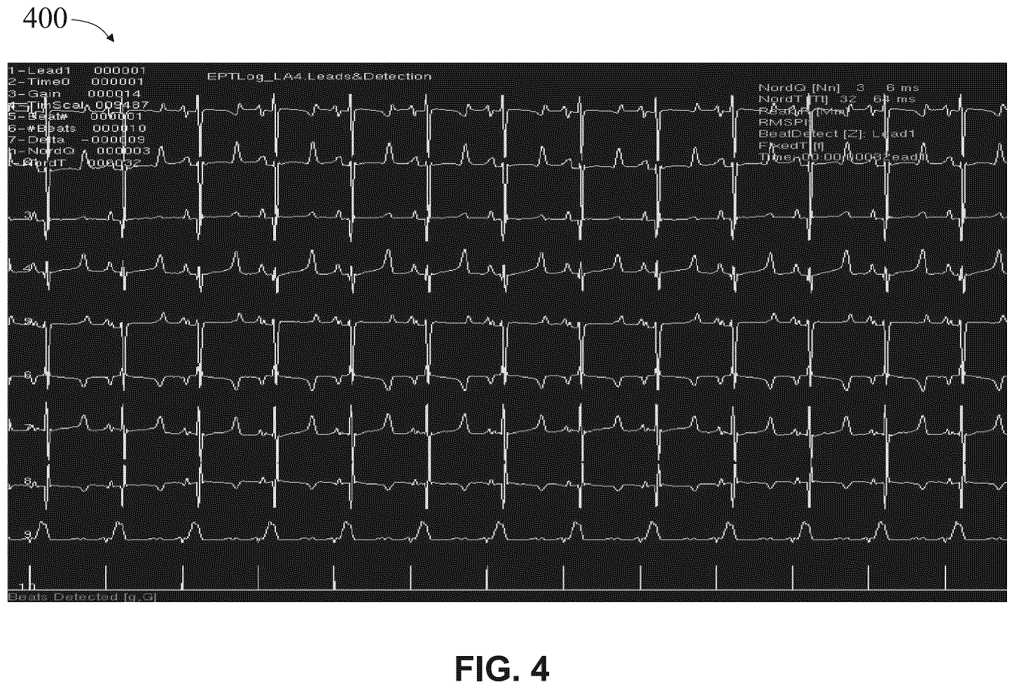

Referring now to FIG. 4, an illustration of an exemplary graph of P wave detection, 400. The graph 400 includes multiple leads, each representing a different perspective of the heart's electrical activity. The graph 400 highlights the detection of P waves, which correspond to the depolarization of the atria. The P wave detection in graph 400 may be achieved using a beat detection algorithm. The algorithm may process the potential signals from each lead to identify the characteristic features of the P wave, such as amplitude, duration, and shape. The detected P waves may be marked on the graph 400, allowing for precise identification and analysis of atrial activity. The beat detection algorithm may filter out noise and artifacts from the potential signals to ensure accurate detection of the P waves. The algorithm may use techniques such as thresholding, pattern recognition, and frequency analysis to distinguish the P waves from other components of the ECG signal. The detected P waves may then use to create a beat template, which serves as a reference for identifying and comparing individual P waves during electroanatomic mapping. The continuous feedback provided by the beat detection algorithm may ensure that the detected P waves are consistent and reliable. This feedback allows for real-time adjustments and refinements to the detection process, improving the overall accuracy and reliability of the electroanatomic mapping. The graph 400 may provide a visual representation of the detected P waves, enabling clinicians to analyze the atrial activity and identify any abnormalities or arrhythmias.

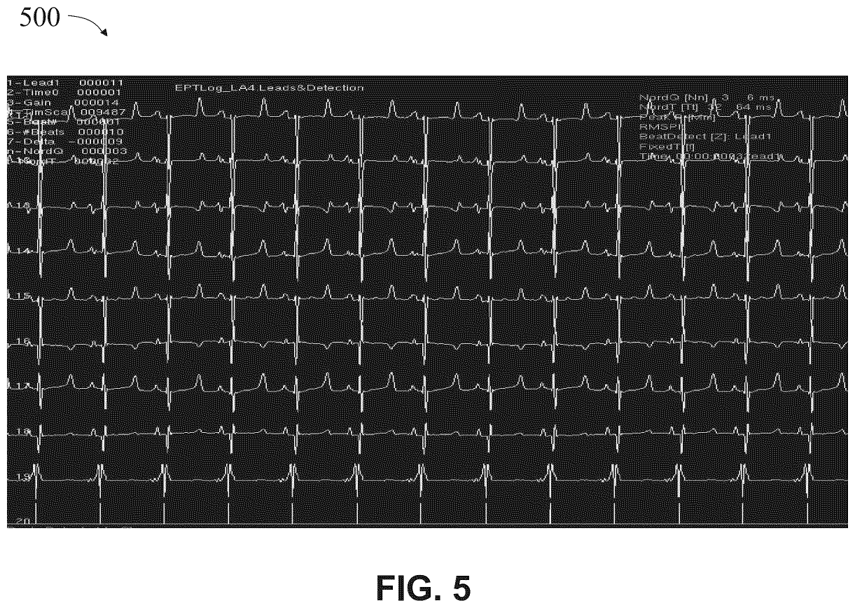

Referring now to FIG. 5, an illustration of an exemplary graph of QRS wave detection, 500. The graph 500 highlights the detection of QRS waves, which correspond to the depolarization of the ventricles. The QRS wave detection in graph 500 may be achieved using a beat detection algorithm. The algorithm may process the potential signals from each lead to identify the characteristic features of the QRS wave, such as amplitude, duration, and shape. The detected QRS waves may be marked on the graph 500, allowing for precise identification and analysis of ventricular activity. The beat detection algorithm may filter out noise and artifacts from the potential signals to ensure accurate detection of the QRS waves. The algorithm may use techniques such as thresholding, pattern recognition, and frequency analysis to distinguish the QRS waves from other components of the ECG signal. The detected QRS waves may then be used to create a beat template, which serves as a reference for identifying and comparing individual QRS waves during electroanatomic mapping. The continuous feedback provided by the beat detection algorithm may ensure that the detected QRS waves are consistent and reliable. This feedback may allow for real-time adjustments and refinements to the detection process, improving the overall accuracy and reliability of the electroanatomic mapping. The graph 500 may provide a visual representation of the detected QRS waves, enabling clinicians to analyze the ventricular activity and identify any abnormalities or arrhythmias.

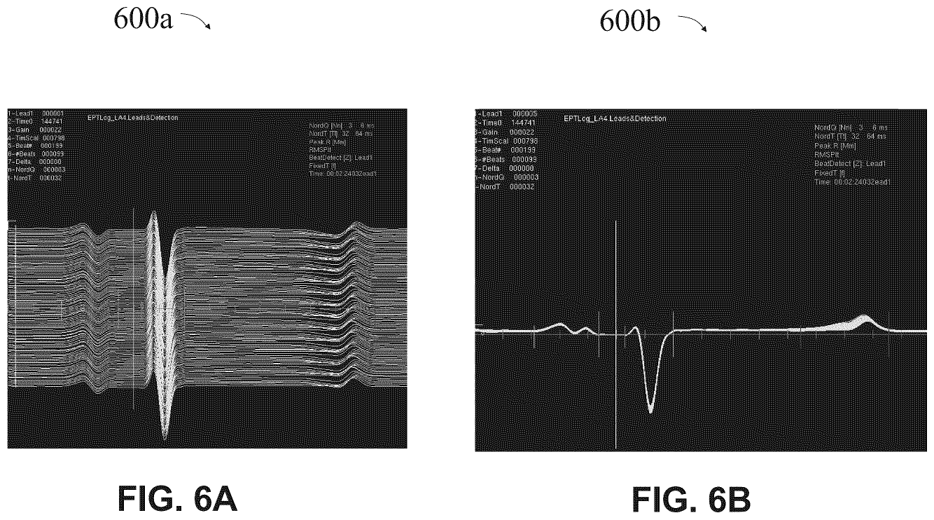

Referring now to FIG. 6A, an illustration of 100 detected beats with the time variable aligned, 600a, and FIG. 6B, an illustration of a single detected beat, 600b. FIG. 6A shows a continuous, rolling, stacked array of detected ECG beats from one lead, with 100 detected and time-aligned beats, the newest on the bottom. This visualization may allow for the assessment of beat morphology stability over time, ensuring that the detected beats are consistent and reliable. In a non-limiting example, the beat detection algorithm may use this visualization to provide continuous feedback on the stability of the detected beats. The algorithm may highlight any deviations in the morphology of the beats, allowing for real-time adjustments and refinements to the detection process. This may ensure that the detected beats are accurately represented in both the geometric and electrical aspects of the electroanatomic map.

FIG. 6B illustrates a single detected beat, providing a detailed view of the beat's morphology. This visualization may allow for the precise analysis of the beat's characteristics, such as the amplitude, duration, and shape of the P wave, QRS complex, and T wave. In a non-limiting example, the beat detection algorithm may use this detailed view to compare the detected beat with the beat template, ensuring accurate detection and mapping of the cardiac event. In a non-limiting example, the continuous feedback provided by the beat detection algorithm may ensure that the detected beats are consistent and reliable. This feedback may allow for real-time adjustments and refinements to the detection process, improving the overall accuracy and reliability of the electroanatomic mapping. The visualizations in FIG. 6A-B may provide a comprehensive tool for clinicians to analyze the detected beats and identify any abnormalities or arrhythmias.

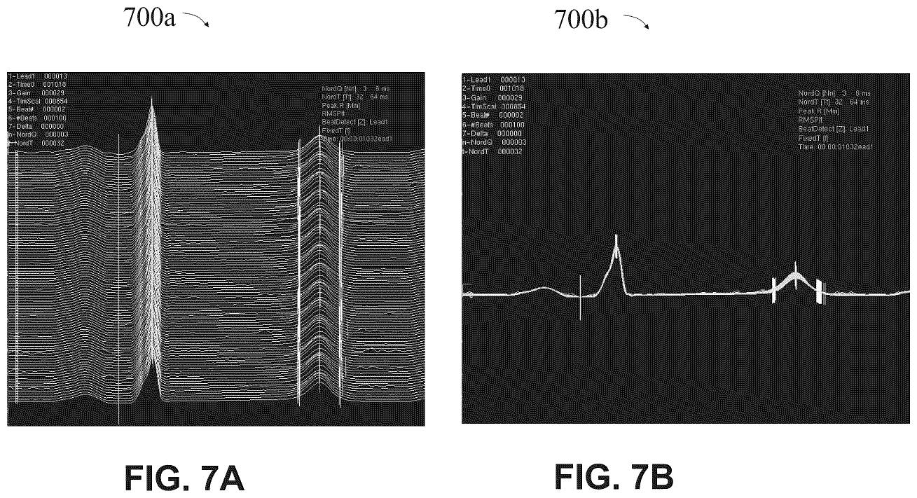

Referring now to FIG. 7A-B, an illustration of a root mean square (RMS) of 12-lead potential signal graph, 700a-b. The RMS calculation may provide a measure of the magnitude of the varying potential signals, which is useful for analyzing the overall signal strength and consistency across multiple leads. The RMS values may be derived from the potential signals detected by the transducers in the catheter during electroanatomic mapping. FIG. 7A shows the RMS values of the 12-lead potential signals over a period. The graph 700a includes multiple traces, each representing the RMS value of a different lead. The traces may provide a visual representation of the signal strength and consistency across the leads, highlighting any variations or anomalies in the potential signals. The RMS calculation may help to identify the overall signal quality and detect any significant deviations that may affect the accuracy of beat detection and mapping. FIG. 7B shows a detailed view of the RMS values for a single lead. The graph 700b provides a closer look at the RMS calculation for one of the 12 leads, allowing for a more precise analysis of the signal strength and consistency for that specific lead. The detailed view may help to identify any localized issues or anomalies in the potential signal, ensuring that the beat detection algorithm can accurately detect and map the cardiac events for that lead. The continuous feedback provided by the RMS calculation may ensure that the detected signals are consistent and reliable, improving the overall accuracy and reliability of the electroanatomic mapping process.

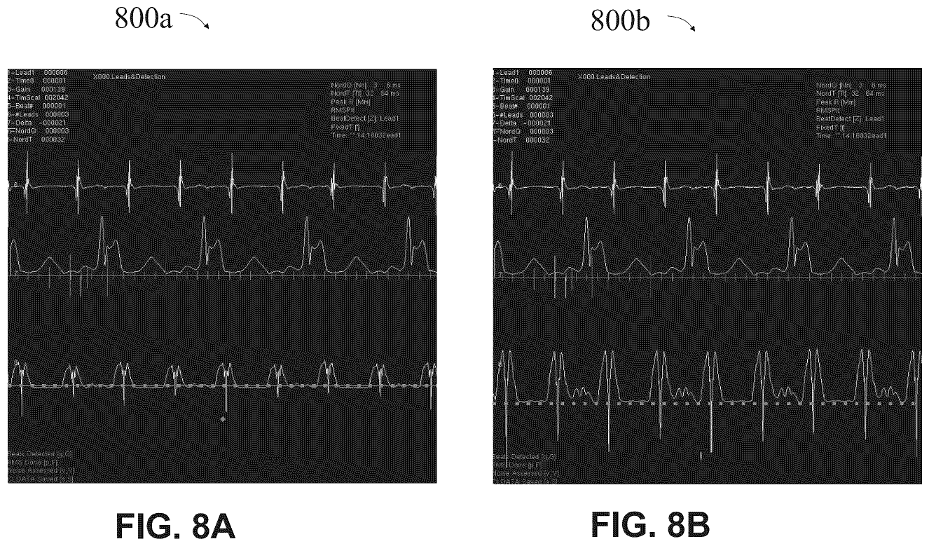

Referring now to FIG. 8A and FIG. 8B illustrate typical atrial flutter (AFL) and reverse typical atrial flutter (AFL) in a 12-lead potential signal graph, respectively. FIGS. 8A-B show the calculation of the derivative of the potential signal for each lead and the summation of these derivatives. FIGS. 8A-B include multiple leads, each representing a different perspective of the heart's electrical activity. In FIG. 8A, the graph 800a may show the typical atrial flutter (AFL) detected in the 12-lead potential signal. The derivative of the potential signal for each lead may be calculated and then summed to highlight the characteristic features of the atrial flutter. The summed derivative signal may provide a clear representation of the atrial flutter's electrical activity, allowing for precise identification and analysis of this cardiac event.

In FIG. 8B, the graph 800b may show the reverse typical atrial flutter (AFL) detected in the 12-lead potential signal. Similar to FIG. 8A, the derivative of the potential signal for each lead may be calculated and then summed to highlight the characteristic features of the reverse atrial flutter. The summed derivative signal may provide a clear representation of the reverse atrial flutter's electrical activity, allowing for precise identification and analysis of this cardiac event. The beat detection algorithm may process the potential signals from each lead to calculate the derivatives and sum them, ensuring accurate detection and mapping of the atrial flutter events. The continuous feedback provided by the beat detection algorithm may ensure that the detected atrial flutter events are consistent and reliable, improving the overall accuracy and reliability of the electroanatomic mapping process.

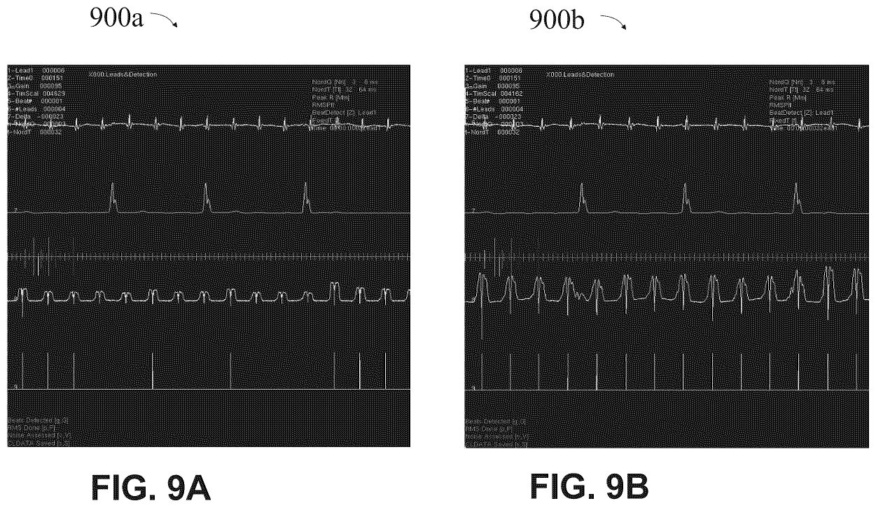

Referring now to FIG. 9A-B, an illustration of a reverse typical atrial flutter (AFL) in a 12-lead potential signal graph where a derivative of the potential signal for each lead is calculated and then summed, 900a-b. FIG. 9A-B show the calculation of the derivative of the potential signal for each lead and the summation of these derivatives. FIG. 9A and FIG. 9B may include multiple leads, each representing a different perspective of the heart's electrical activity. FIG. 9A may show the reverse typical atrial flutter detected in the 12-lead potential signal. The derivative of the potential signal for each lead may be calculated and then summed to highlight the characteristic features of the reverse atrial flutter. The summed derivative signal may provide a clear representation of the reverse atrial flutter's electrical activity, allowing for precise identification and analysis of this cardiac event.

FIG. 9B may show another instance of reverse typical atrial flutter detected in the 12-lead potential signal. Similar to FIG. 9A, the derivative of the potential signal for each lead may be calculated and then summed to highlight the characteristic features of the reverse atrial flutter. The summed derivative signal may provide a clear representation of the reverse atrial flutter's electrical activity, allowing for precise identification and analysis of this cardiac event. The beat detection algorithm may process the potential signals from each lead to calculate the derivatives and sum them, ensuring accurate detection and mapping of the atrial flutter events. The continuous feedback provided by the beat detection algorithm may ensure that the detected atrial flutter events are consistent and reliable, improving the overall accuracy and reliability of the electroanatomic mapping process.

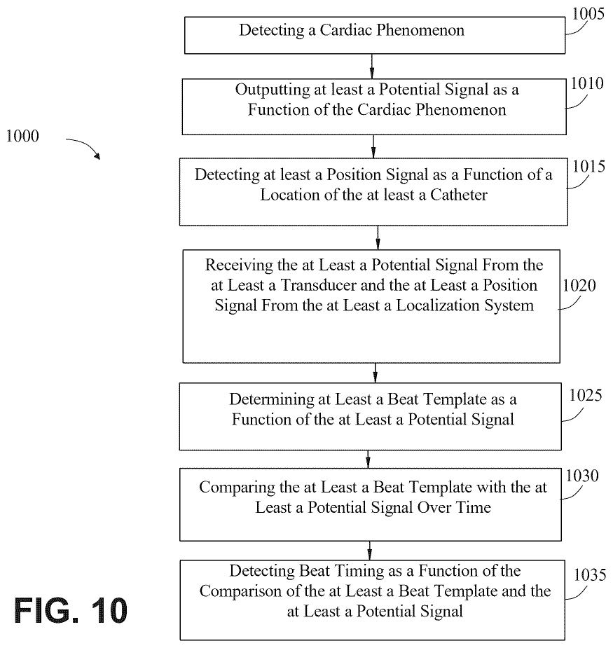

Referring now to FIG. 10, a flow diagram of an exemplary method 1000 for automatic beat detection during electro anatomical mapping is illustrated. At step 1005, method 1000 includes detecting, using at least a catheter configured for intracardiac use and comprising at least a transducer, a cardiac phenomenon. This may be implemented as described and with reference to FIGS. 1-9.

Still referring to FIG. 10, at step 1010, method 1000 includes outputting, by the at least a catheter, at least a potential signal as a function the cardiac phenomenon. This may be implemented as described and with reference to FIGS. 1-9.

Still referring to FIG. 10, at step 1015, method 1000 includes detecting, using at least a localization system, at least a position signal as a function of a location of the at least a catheter. This may be implemented as described and with reference to FIGS. 1-9.

Still referring to FIG. 10, at step 1020, method 1000 includes receiving, using the at least a processor, the at least a potential signal from the at least a transducer and the at least a position signal from the at least a localization system. This may be implemented as described and with reference to FIGS. 1-9.

Still referring to FIG. 10, at step 1025, method 1000 includes determining, using a beat detection algorithm, at least a beat template as a function of the at least a potential signal. This may be implemented as described and with reference to FIGS. 1-9.

Still referring to FIG. 10, at step 1030, method 1000 includes comparing, using the beat detection algorithm, the at least a beat template with the at least a potential signal over time. This may be implemented as described and with reference to FIGS. 1-9.

Still referring to FIG. 10, at step 1035, method 1000 includes detecting, using the beat detection algorithm, beat timing as a function of the comparison of the at least a beat template and the at least a potential signal. This may be implemented as described and with reference to FIGS. 1-9.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding may readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

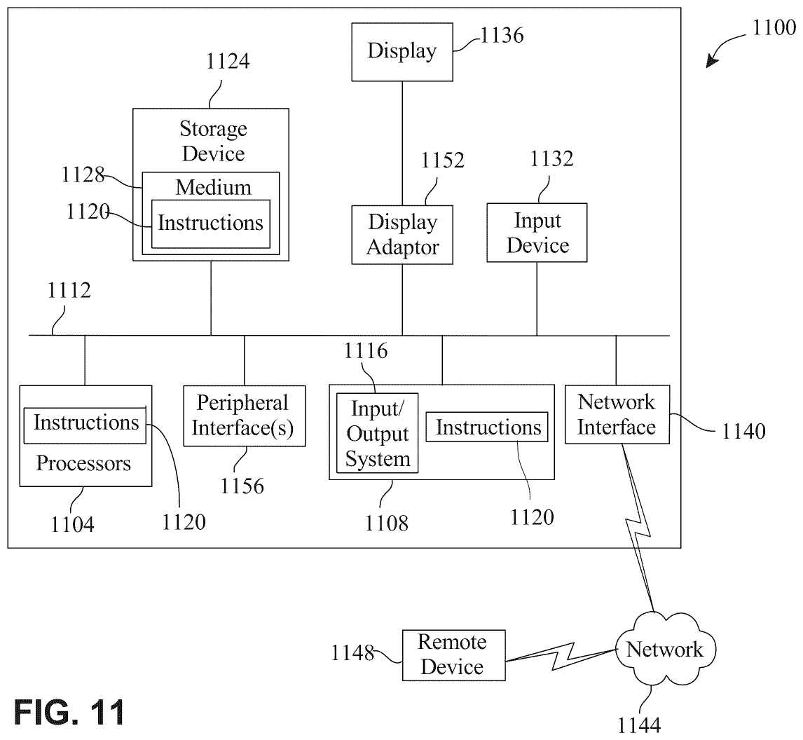

FIG. 11 shows a diagrammatic representation of one embodiment of computing device in the exemplary form of a computer system 1100 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1100 includes a processor 1104 and a memory 1108 that communicate with each other, and with other components, via a bus 1112. Bus 1112 may include any of several types of bus structures including, but not limited to, a memory bus, a memory controller, a peripheral bus, a local bus, and any combinations thereof, using any of a variety of bus architectures.

Processor 1104 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 1104 may be organized according to Von Neumann and/or Harvard architecture as a non-limiting example. Processor 1104 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), system on module (SOM), and/or system on a chip (SoC).

Memory 1108 may include various components (e.g., machine-readable media) including, but not limited to, a random-access memory component, a read only component, and any combinations thereof. In one example, a basic input/output system 1116 (BIOS), including basic routines that help to transfer information between elements within computer system 1100, such as during start-up, may be stored in memory 1108. Memory 1108 may also include (e.g., stored on one or more machine-readable media) instructions (e.g., software) 1120 embodying any one or more of the aspects and/or methodologies of the present disclosure. In another example, memory 1108 may further include any number of program modules including, but not limited to, an operating system, one or more application programs, other program modules, program data, and any combinations thereof.

Computer system 1100 may also include a storage device 1124. Examples of a storage device (e.g., storage device 1124) include, but are not limited to, a hard disk drive, a magnetic disk drive, an optical disc drive in combination with an optical medium, a solid-state memory device, and any combinations thereof. Storage device 1124 may be connected to bus 1112 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 1124 (or one or more components thereof) may be removably interfaced with computer system 1100 (e.g., via an external port connector (not shown)). Particularly, storage device 1124 and an associated machine-readable medium 1128 may provide nonvolatile and/or volatile storage of machine-readable instructions, data structures, program modules, and/or other data for computer system 1100. In one example, software 1120 may reside, completely or partially, within machine-readable medium 1128. In another example, software 1120 may reside, completely or partially, within processor 1104.

Computer system 1100 may also include an input device 1132. In one example, a user of computer system 1100 may enter commands and/or other information into computer system 1100 via input device 1132. Examples of an input device 1132 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1132 may be interfaced to bus 1112 via any of a variety of interfaces (not shown) including, but not limited to, a serial interface, a parallel interface, a game port, a USB interface, a FIREWIRE interface, a direct interface to bus 1112, and any combinations thereof. Input device 1132 may include a touch screen interface that may be a part of or separate from display 1136, discussed further below. Input device 1132 may be utilized as a user selection device for selecting one or more graphical representations in a graphical interface as described above.

A user may also input commands and/or other information to computer system 1100 via storage device 1124 (e.g., a removable disk drive, a flash drive, etc.) and/or network interface device 1140. A network interface device, such as network interface device 1140, may be utilized for connecting computer system 1100 to one or more of a variety of networks, such as network 1144, and one or more remote devices 1148 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1144, may employ a wired and/or a wireless mode of communication. In general, any network topology may be used. Information (e.g., data, software 1120, etc.) may be communicated to and/or from computer system 1100 via network interface device 1140.

Computer system 1100 may further include a video display adapter 1152 for communicating a displayable image to a display device, such as display 1136. Examples of a display device include, but are not limited to, a liquid crystal display (LCD), a cathode ray tube (CRT), a plasma display, a light emitting diode (LED) display, and any combinations thereof. Display adapter 1152 and display 1136 may be utilized in combination with processor 1104 to provide graphical representations of aspects of the present disclosure. In addition to a display device, computer system 1100 may include one or more other peripheral output devices including, but not limited to, an audio speaker, a printer, and any combinations thereof. Such peripheral output devices may be connected to bus 1112 via a peripheral interface 1156. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. An apparatus for automatic beat detection during electroanatomical mapping, wherein the apparatus comprises:
at least a catheter configured for intracardiac use, the at least a catheter comprising at least a transducer configured to detect a cardiac phenomenon and output at least a potential signal, as a function of the cardiac phenomenon;
at least a localization system configured to detect at least a position signal as a function of a location of the at least a catheter;
at least a computing device, wherein the computing device comprises:
a memory; and
at least a processor communicatively connected to the memory, wherein the memory contains instructions configuring the at least a processor to:
receive the at least a potential signal from the at least a transducer and the at least a position signal from the at least a localization system;
determine, using a beat detection algorithm, at least a beat template of a plurality of beat templates as a function of the at least a potential signal;
compare, using the beat detection algorithm, the at least a beat template with the at least a potential signal over time, wherein comparing further comprises employing a pattern recognition algorithm configured to extract at least a feature from the at least a beat template;
detect, using the beat detection algorithm, beat timing as a function of a comparison of the at least a beat template and the at least a potential signal;
provide continuous feedback to a user through a display device, wherein the continuous feedback comprises information on beat morphology stability derived from the beat timing; and
assess the beat morphology stability by comparing waveform characteristics of consecutive beats for consistency; and
a notification system communicatively connected to the at least a computing device, wherein the notification system is configured to generate at least an alert as a function of the assessment of the beat morphology stability thereby allowing for real-time component adjustment, wherein the at least an alert comprises at least one of a visual alert and an audio alert.

2. The apparatus of claim 1, wherein the at least a localization system comprises an electromagnetic localization system.

3. The apparatus of claim 1, wherein the at least a potential signal comprises electrocardiogram data.

4. The apparatus of claim 1, wherein the beat detection algorithm is further configured to:
calculate a derivative of a plurality of potential signals, wherein the plurality of potential signals comprises a potential signal for each lead of a plurality of leads;
sum the derivatives the plurality of potential signals to determine a summed potential signal;
identify a local minimum derivative value of the summed potential signal; and
compare, using the local minimum derivative value, the summed potential signal with the at least a beat template.

5. The apparatus of claim 1, comprising a potential signal filter, wherein the potential signal filter is configured to:
detect at least a noise in the at least a potential signal; and
remove the at least a noise from the at least a potential signal.

6. The apparatus of claim 1, comprising a motion filter, wherein the at least a processor is further configured to:
detect, using the motion filter, a cardiac motion and a respiratory motion; and
remove, using the motion filter, the cardiac motion and the respiratory motion from the at least a position signal.

7. (canceled)

8. The apparatus of claim 1, wherein the beat detection algorithm is further configured to match a first template of the at least a beat template and a second template of the at least a beat template with the at least a potential signal as a function of the beat timing.

9. The apparatus of claim 8, wherein the first template comprises a P wave template and the second template comprises a QRS wave template.

10. The apparatus of claim 8, wherein the plurality of beat templates comprises a P wave template, a QRS wave template, and premature ventricular contraction wave template.

11. A method for automatic beat detection during electroanatomical mapping, wherein the method comprises:
detecting, using at least a catheter configured for intracardiac use and comprising at least a transducer, a cardiac phenomenon;
outputting, by the at least a catheter, at least a potential signal as a function the cardiac phenomenon;
detecting, using at least a localization system, at least a position signal as a function of a location of the at least a catheter;
receiving, using at least a processor, the at least a potential signal from the at least a transducer and the at least a position signal from the at least a localization system;
determining, by the at least a processor and using a beat detection algorithm, at least a beat template of a plurality of beat templates as a function of the at least a potential signal;
comparing, using the beat detection algorithm, the at least a beat template with the at least a potential signal over time, wherein comparing further comprises employing a pattern recognition algorithm configured to extract at least a feature from the at least a beat template;
detecting, using the beat detection algorithm, beat timing as a function of a comparison of the at least a beat template and the at least a potential signal;
providing, using the at least a processor, continuous feedback to a user through a display device, wherein the continuous feedback comprises information on beat morphology stability derived from the beat timing;
assessing, using the at least a processor, the beat morphology stability by comparing waveform characteristics of consecutive beats for consistency; and
generating, using a notification system communicatively connected to the at least a computing device, at least an alert as a function of the assessment of the beat morphology stability thereby allowing for real-time component adjustment, wherein the at least an alert comprises at least one of a visual alert and an audio alert.

12. The method of claim 11, wherein the at least a localization system comprises an electromagnetic localization system.

13. The method of claim 11, wherein the at least a potential signal comprises a plurality of electrocardiogram data.

14. The method of claim 11, further comprising:
calculating, using the beat detection algorithm, a derivative of the potential signal for each lead;
summing, using the beat detection algorithm, the derivative of the potential signal for each lead;
identifying, using the beat detection algorithm, a local minimum derivative value of the summed potential signal; and
comparing, using the beat detection algorithm, using the local minimum derivative value, the summed potential signal with the at least a beat template.

15. The method of claim 11, further comprising:
detecting, using a potential signal filter, at least a noise in the at least a potential signal; and
removing, using the potential signal filter, the at least a noise from the at least a potential signal.

16. The method of claim 11, further comprising a motion filter,
wherein the motion filter is configured to:
detect, using the at least a processor and a motion filter, a cardiac motion and a respiratory motion; and
remove, using the at least a processor and the motion filter, the cardiac motion and the respiratory motion from the at least a position signal.

17. (canceled)

18. The method of claim 11, further comprising matching, using the beat detection algorithm, a first template of the at least a beat template and a second template of the at least a beat template with the at least a potential signal as a function of the beat timing.

19. The method of claim 18, wherein the first template comprises a P wave template and the second template comprises a QRS wave template.

20. The method of claim 18, wherein the plurality of beat templates comprises a P wave template, a QRS wave template, and premature ventricular contraction wave template.
